(19) United States
(12) Patent Application Publication
(10) Pub. No.: US 2011/0233487 A1
(43) Pub. Date: Sep. 29, 2011

(54) Synthesis of Chalcogenide Ternary and Quaternary Nanotubes Through Directed Compositional Alterations of Bacterial As-S Nanotubes

(75) Inventors: Hor Gil Hur, Gwangju (KR); Jiang Sheng Hua, Gwangju (KR); Nosang V. Myung, (US)

(73) Assignee: Gwangju Institute of Science and Technology, Gwangju (KR)

(21) Appl. No.: 12/968,954

(22) Filed: Dec. 15, 2010

(30) Foreign Application Priority Data
Mar. 3, 2010 (KR) 10-2010-0024913

(51) Int. Cl. H01B 1/10; C01B 19/00; C01G 28/00; C12P 3/00; B82Y 40/00; B82Y 30/00

(52) U.S. Cl. 252/519.4; 423/508; 423/511; 435/168; 977/896; 977/762

(57) ABSTRACT

Provided is a method for preparing a chalcogenic hybrid nanostructure including: (a) adding a chalcogenic nanostructure, an electron donor and an electron acceptor to a medium containing metal-reducing bacteria to prepare a reaction mixture, the electron acceptor including a chalcogen element; and (b) performing a metal reduction reaction using the prepared reaction mixture to prepare a chalcogenic hybrid nanostructure with the chalcogen element of the electron acceptor incorporated. The present disclosure provides a new method allowing preparation of a chalcogenic hybrid nanostructure comprising three or more components using metal-reducing bacteria. The disclosure allows preparation of a nanostructure in a more economical and eco-friendly manner. The disclosure also allows control of morphological, physical/chemical and electrical properties of the prepared nanostructure. In addition, the present disclosure provides a nanomaterial that can be useful in nanoelectronic and optoelectronic devices.

### CROSS-REFERENCE TO RELATED APPLICATION

This application claims priority from prior foreign patent application 10-2010-0024913, filed Mar. 3, 2010, in the Republic of Korea.

### BACKGROUND OF THE INVENTION

1. Field of the Invention

The inventors have found out that dissimilatory metal-reducing bacteria can contribute to the preparation of chalcogenic hybrid nanostructures, such as ternary or quaternary chalcogenic nanostructures, and established a protocol for the preparation thereof. Further, they have found out that the morphological, physical/chemical and electrical properties of the chalcogenic hybrid nanostructures can be tuned by the preparation method, and that the resulting nanostructures, e.g. nanotube, may be utilized for nanoelectronic devices, optoelectronic devices or solar cells.

The present disclosure is directed to providing a method for preparing a chalcogenic hybrid nanostructure.

The present disclosure is also directed to providing a chalcogenic hybrid nanostructure.

Other features and aspects will be apparent from the following detailed description, the drawings, and the claims.

2. Description of the Related Art

Semiconducting nanostructures have become intensively investigated by both experimentalists and theoreticians because of their unique size dependent electronic and optical properties¹. One group of the most investigated semiconductors is chalcogenide compounds (MX, M=As, Cd, Zn; X═S, Se, Te) because their band gap can be easily fine-tuned from zero (like the semi-metal HgTe) to large band gap (e.g. ZnS (E_{g}=3.8 eV))². In addition to composition, the properties of chalcogenide can be further “tuned” by controlling the dimension of materials in nanoscale. Since the first discovery of carbon nanotubes (CNTs) in 1991³, diverse organic and inorganic one-dimensional (1D) nanostructures including the semiconducting nanowires and nanotubes have been synthesized⁴ and used as important building blocks for many potential applications^{3,5,6}.

However, majority of the nanostructures were synthesized through chemical or physical methods which typically require harsh reaction conditions such as high operating temperature, extremely high or ultra-low pressure, catalyst and toxic precursors⁷. In contrast, bio-inspired or biomimic routes allow synthesizing nanoengineered materials with “greener” precursors under mild ambient conditions. It is well-known that microorganisms play essential roles in the biogeochemical cycling of elements and in the formation of unique minerals/materials⁸⁻¹⁰ through altering the valence/oxidation state of heavy metals and metalloids for anaerobic respiration¹¹⁻¹³. Recent researches have showed new insight on the reducing capabilities of certain anaerobic bacteria which offer significant utility in both heavy metal remediation and nano-manufacturing^{14, 15}. Among the bacteria, dissimilatory metal-reducing bacteria have shown to contribute to the formation of diverse nano-scaled minerals by virtue of their respiring fashion^{4, 16, 17}. Interestingly, Shewanella sp. HN-41 showed the biological synthesis of one-dimensional As—S nanotubes which exhibited photoactive and semiconducting properties via reduction of As(V) and thiosulfate under ambient anaerobic culture conditions. In addition, Shewanella sp. HN-41 has the ability to reduce selenite (Se(IV)) to elemental selenium, forming amorphous Se nanospheres^{16, 18}.

It has been reported that diverse semiconducting inorganic hybrid nanotubes were synthesized via ion exchange reaction to enhance the functionality and applicability⁹⁻²¹. It is also known that electrical conduction is closely associated with the structures such as the grain size, defects and impurities. Especially, the conduction of semiconductors is mainly governed by the grain boundary scattering where amorphous/nanocrystalline materials have much lower carrier concentration and mobility than single or polycrystalline materials with larger grains²². As the grain size increased, the contribution of grain resistance would be reduced, resulting in smaller thermal activation energy, E_{A}²³. This suggested that the biological photoactive As—S nanotubes can be transformed into tunable structure with varying composition and ideal electrical property via kinetically controlled solution-phase ion exchange reaction and crystallization.

Thus, in this study, various biological activities of dissimilatory metal-reducing bacteria, including formation of the selenium nanoparticles from Se(IV) reduction and the photoactive As—S nanotubes, were applied for synthesis of the versatile ternary and quaternary chalcogenide (i.e. As—S—Se, As—Cd—S and As—Cd—S—Se) nanotubes with aid of biological and/or abiological activities. Se and/or Cd were incorporated either by biogenic deposition or ion exchange onto As—S nanotubes to control their electrical properties, which may open-up the possibility to integrate these nanotubes in nanoelectronics, optoelectronics, and solar cells. The mineralogical, crystal structure, morphology and electrical properties of nanotubes were characterized, thereby understanding the influence of the ratio and different elemental composition.

Throughout this application, various patents and publications are referenced, and citations are provided in parentheses. The disclosure of these patents and publications in their entities are hereby incorporated by references into this application in order to more fully describe this invention and the state of the art to which this invention pertains.

### SUMMARY OF THE INVENTION

The inventors have found out that dissimilatory metal-reducing bacteria can contribute to the preparation of chalcogenic hybrid nanostructures, such as ternary or quaternary chalcogenic nanostructures, and established a protocol for the preparation thereof. Further, they have found out that the morphological, physical/chemical and electrical properties of the chalcogenic hybrid nanostructures can be tuned by the preparation method, and that the resulting nanostructures, e.g. nanotube, may be utilized for nanoelectronic devices, optoelectronic devices or solar cells.

The present disclosure is directed to providing a method for preparing a chalcogenic hybrid nanostructure.

The present disclosure is also directed to providing a chalcogenic hybrid nanostructure.

Other features and aspects will be apparent from the following detailed description, the drawings, and the claims.

### BRIEF DESCRIPTION OF THE DRAWINGS

FIG. 1 Concentration of As and Se in the medium containing Shewnella sp. HN-41 (a), SEM image (b), TEM image (c), line-scan EDX profile across the cross section (d), TEM image with SAED pattern of As—S—Se nanotubes (e), and X-ray diffraction patterns (f).

FIG. 2 Concentration of As and Se in the liquid phase containing Shewnella sp. HN-41 (a), SEM image (b), TEM image (c), line-scan EDX profile across the cross section (d), TEM image with SAED pattern of As—Cd—S nanotubes (e), and X-ray diffraction patterns (f).

FIG. 3 Concentration of As and Se in the medium containing Shewnella sp. HN-41 (a), SEM image (b), TEM image (c), line-scan EDX profile across the cross section (d), TEM image with SAED pattern of As—Cd—S—Se nanotubes (e), and X-ray diffraction patterns (f).

FIG. 4 Temperature dependent I-V curves (a), resistance change as a function of temperature (b), transfer characteristics of As—Cd—S nanotubes with inset figure of aligned As—Cd—S nanotubes between electrode pads (c), grain size vs. thermal activation energy (d) and field effect mobility vs. carrier concentration of As—S, As—Cd—S, As—S—Se, and As—Cd—S—Se nanotubes (e). (As—S and As—S—Se have amorphous phase)

FIG. 5 Size distributions of As—S (a), As—S—Se (b), As—Cd—S (c), and As—Cd—S—Se (d) nanotubes. Numbers of the nanotubes counted, averages and standard deviations of the diameters of the nanotubes are shown on the diagrams. Solid lines: Estimation by Gaussian fitting.

FIG. 6 Concentration of As, Cd and Se in the HNO₃ solutions which digested As—S—Se (a), As—Cd—S (b), and As—Cd—S—Se (c) nanotubes, respectively.

FIG. 7 TEM images of Se nodules attached to the As—S nanotubes synthesized in the presence of Se(IV) with no bacteria after purification of the As—S nanotubes (a), no-uniformed As—S—Se nanotubes formed after direct addition of Se(IV) into the As—S producing medium in the presence of strain HN-41 (b), and As—Cd—S nanotubes formed via Cd—As ion exchange reaction for 8 h (c) after purification of the As—S nanotubes.

FIG. 8 HR-TEM image (a) and FFT with the analyzed compositions (b) for the As—Cd—S nanotubes, and HR-TEM image (c) and FFT with the analyzed compositions (d) for the As—Cd—S—Se nanotubes.

FIG. 9 Temperature dependent I-V curves (a), resistance change as a function of temperature (b), and transfer characteristics of As—S nanotubes with inset figure of aligned As—S nanotubes between electrode pads (c).

FIG. 10 Temperature dependent I-V curves (a), resistance change as a function of temperature (b), and transfer characteristics of As—S—Se nanotubes with inset figure of aligned As—S—Se nanotubes between electrode pads (c).

FIG. 11 Temperature dependent I-V curves (a), resistance change as a function of temperature (b), and transfer characteristics of As—Cd—S—Se nanotubes with inset figure of aligned As—Cd—S—Se nanotubes between electrode pads (c).

### DETAILED DESCRIPTION OF THE INVENTION

The advantages, features and aspects of the present disclosure will become apparent from the following description of the embodiments with reference to the accompanying drawings, which is set forth hereinafter. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art. The terminology used herein is for the purpose of describing particular embodiments only and is not intended to be limiting of the example embodiments. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Hereinafter, exemplary embodiments will be described in detail with reference to the accompanying drawings.

In an aspect, the present disclosure provides a method for preparing a chalcogenic hybrid nanostructure comprising: (a) adding a chalcogenic nanostructure, an electron donor and an electron acceptor to a medium containing metal-reducing bacteria to prepare a reaction mixture, the electron acceptor comprising a chalcogen element; and (b) performing a metal reduction reaction using the prepared reaction mixture to prepare a chalcogenic hybrid nanostructure with the chalcogen element of the electron acceptor incorporated.

The inventors have found out that dissimilatory metal-reducing bacteria can contribute to the preparation of chalcogenic hybrid nanostructures, such as ternary or quaternary chalcogenic nanostructures, and established a protocol for the preparation thereof. Further, they have found out that the morphological, physical/chemical and electrical properties of the chalcogenic hybrid nanostructures can be tuned by the preparation method, and that the resulting nanostructures, e.g. nanotube, may be utilized for nanoelectronic devices, optoelectronic devices or solar cells.

One of the important features of the present disclosure is to use metal-reducing bacteria. Specifically, the metal-reducing bacteria may belong to the genus Thauera, Sulfurospirillum, Bacillus, Ralstonia, Desulfotomaculum, Desulfovibrio or Shewanella. These bacteria are known to reduce selenate or selenite to elemental selenium (Se) [Zhang, B., et al., Biomolecule-assisted synthesis of single-crystalline selenium nanowires and nanoribbons via a novel flake-cracking mechanism. Nanotechnology 17: 385-390 (2006); Zhang, H., et al., Selenium nanotubes synthesized by a novel solution phase approach. Journal of Physical Chemistry B 108: 1179-1182 (2004); Zhang, S. Y., et al., Rapid, large-scale synthesis and electrochemical behavior of faceted single-crystalline selenium nanotubes. Journal of Physical Chemistry B 110: 9041-9047 (2006)]. More specifically, the metal-reducing bacteria may belong to the genus Shewanella. Most specifically, the metal-reducing bacteria may be Shewanella sp. HN-41 (KCTC 10837BP).

The medium used to grow the metal-reducing bacteria and maintain their activity may be any medium known in the art. For example, a HEPES-buffered basal medium may be used (Lee J-H, et al., Geomicrobiol. J. 24: 31-41 (2007)). Specifically, the medium may be prepared under an anaerobic condition. For example, the medium may be prepared under an anaerobic condition prepared by boiling followed by 100% N₂ purging.

According to a specific embodiment of the present disclosure, the chalcogenic nanostructure which is used as a precursor may comprise at least one chalcogen element selected from a group consisting of As, Cd, Zn, S, Se and Te. More specifically, it may comprise at least two chalcogen elements. More specifically, it may be a binary chalcogenic nanostructure comprising two chalcogen elements. Most specifically, it may be a binary nanostructure comprising As and S (e.g. an As₂S₃ nanotube).

A specific example of the binary nanostructure comprising As and S, which is used as a precursor in the present disclosure, is the arsenic sulfide (As—S; As₂S₃) nanotube developed by the inventors of the present disclosure using Shewanella sp. HN-41 (KCTC 10837BP) [Lee, J.-H. et al. Biogenic formation of photoactive arsenic-sulfide nanotubes by Shewanella sp. HN-41. Proc. Natl. Acad. Sci. USA 104, 20410-20415 (2007)].

The As—S (As₂S₃) nanotube as the precursor may be prepared by reacting an electron donor (e.g., lactate) and a salt comprising As or S (e.g., thiosulfate or arsenate) as an electron acceptor with metal-reducing bacteria (most specifically, Shewanella sp. HN-41 (KCTC 10837BP)) under an appropriate condition (e.g., at 30° C. in the dark).

The chalcogenic nanostructure as the precursor may have various nanosturcures. According to a specific embodiment of the present disclosure, the chalcogenic nanostructure as the precursor may be a nanotube, a nanowire, a nanoneedle, a nanoribbon, a nanorod, a pulverized nanowire, a nanotetrapod, a nanotripod, a nanobipod, a nanocrystal, a nanodot, a quantum dot or a nanoparticle. More specifically, it may be a nanotube or a nanowire. Most specifically, it may be a nanotube.

As used herein, the term “nanostructure” refers to a structure having a diameter of 500 nm or smaller, specifically 400 nm or smaller, more specifically 200 nm or smaller, further more specifically 100 nm or smaller, most specifically 60 nm or smaller.

The electron donor used in the step (a) is not particularly limited. Specifically it may be an electron donor in salt form. For example, the electron donor used in the step (a) may be lactate.

The electron acceptor the step (a) is an electron acceptor comprising a chalcogen element further incorporated in addition to that of the chalcogenic nanostructure precursor. Specifically, the electron acceptor comprising the chalcogen element may be a salt comprising a chalcogen element in oxidized state. More specifically, it may be a salt of Se (e.g., a salt of Se(IV)). For example, if Se is the chalcogen element further incorporated in addition to the chalcogen element of the chalcogenic nanostructure precursor, a selenite (e.g., sodium selenite) may be used as the electron acceptor.

After the reaction mixture is prepared, a metal reduction reaction is performed using the prepared reaction mixture to prepare a chalcogenic hybrid nanostructure with the chalcogen element of the electron acceptor incorporated. Specifically, the finally prepared chalcogenic hybrid nanostructure may be a nanotube, a nanowire, a nanoneedle, a nanoribbon, a nanorod, a pulverized nanowire, a nanotetrapod, a nanotripod, a nanobipod, a nanocrystal, a nanodot, a quantum dot or a nanoparticle. More specifically, it may be a nanotube or a nanowire. Most specifically, it may be a nanotube.

The metal reduction reaction using the metal-reducing bacteria may be performed by incubation in the dark specifically at 20-40° C., more specifically 25-35° C., most specifically 30° C.

According to a specific embodiment of the present disclosure, the electron acceptor comprising the chalcogen element may be a salt of Se, and the prepared chalcogenic hybrid nanostructure may be a ternary nanostructure comprising As, S and Se.

According to a specific embodiment of the present disclosure, as a result of the metal reduction reaction using the metal-reducing bacteria, the chalcogen element of the electron acceptor is incorporated into the chalcogenic nanostructure through replacement rather than through deposition.

More specifically, the chalcogenic nanostructure as the precursor may be a binary nanostructure comprising As and S, and the chalcogen element of the electron acceptor may be incorporated into the chalcogenic nanostructure by partially replacing S through replacement rather than through deposition. Specifically, Se of an Se salt may be incorporated into the chalcogenic nanostructure by partially replacing S through replacement rather than through deposition to give a ternary chalcogenic hybrid nanostructure.

Most specifically, the chalcogenic hybrid nanostructure prepared according to the present disclosure may be a ternary nanostructure represented by As₂SₓSe₃₋ₓ ((0<x<3).

According to a specific embodiment of the present disclosure, the method further comprises, after the step (b): adding a medium containing metal-reducing bacteria, an electron donor and a chalcogen element-containing electron acceptor to the prepared chalcogenic hybrid nanostructure to prepare a reaction mixture; and performing a metal reduction reaction to prepare a second chalcogenic hybrid nanostructure with the chalcogen element of the electron acceptor incorporated.

In another aspect, the present disclosure provides a method for preparing a chalcogenic hybrid nanostructure comprising: (a) preparing a reaction mixture comprising a chalcogenic nanostructure and a chalcogen element-containing salt; and (b) performing an ion-exchange reaction using the prepared reaction mixture to prepare a chalcogenic hybrid nanostructure with the chalcogen element of the chalcogen element-containing salt incorporated.

According to this method, the chalcogenic hybrid nanostructure is prepared chemically through an ion-exchange reaction without using bacteria.

The chalcogenic nanostructure as a precursor may be one synthesized biogenically using metal-reducing bacteria. The chalcogenic nanostructure may be synthesized biogenically using metal-reducing bacteria according to the above-described method.

According to a specific embodiment of the present disclosure, the chalcogenic nanostructure as the precursor may comprise at least one chalcogen element selected from a group consisting of As, Cd, Zn, S, Se and Te. More specifically, the chalcogenic nanostructure may be a binary nanostructure comprising As and S.

According to a specific embodiment of the present disclosure, the chalcogen element-containing salt may be a salt comprising a chalcogen element in oxidized state.

The ion-exchange reaction using the reaction mixture comprising the chalcogenic nanostructure and the chalcogen element-containing salt may be performed in the dark at an appropriate temperature (specifically at 20-40° C., more specifically 25-35° C. and most specifically 30° C.).

According to a specific embodiment of the present disclosure, the chalcogen element-containing salt may be a salt of Cd, and the prepared chalcogenic hybrid nanostructure may be a ternary nanostructure comprising As, Cd and S.

According to a specific embodiment of the present disclosure, either or both of the chalcogenic nanostructure and the chalcogenic hybrid nanostructure may be a nanotube or a nanowire.

According to a specific embodiment of the present disclosure, the chalcogenic nanostructure may be a binary nanostructure comprising As and S, and the chalcogen element of the chalcogen element-containing salt may be incorporated into the chalcogenic nanostructure by partially replacing As through cation-exchange reaction.

According to a specific embodiment of the present disclosure, the chalcogenic hybrid nanostructure may be represented by As₂₋ₓCdₓS₃ (0<x<2). More specifically, the chalcogenic hybrid nanostructure represented by As₂₋ₓCdₓS₃ (0<x<2) may have p-type semiconductor properties.

According to a specific embodiment of the present disclosure, the method further comprises, after the step (b): adding a medium containing metal-reducing bacteria, an electron donor and a chalcogen element-containing electron acceptor to the prepared chalcogenic hybrid nanostructure to prepare a reaction mixture; and performing a metal reduction reaction to prepare a second chalcogenic hybrid nanostructure with the chalcogen element of the electron acceptor incorporated.

In this way, a quaternary hybrid nanostructure may be prepared from a ternary hybrid nanostructure by further incorporating a chalcogen element.

The process for preparing the second chalcogenic hybrid nanostructure is the same as the biogenic process using the metal-reducing bacteria described above. Thus, detailed description thereof will be omitted to avoid unnecessarily obscuring the present disclosure.

According to a specific embodiment of the present disclosure, in the preparation of the second hybrid nanostructure, the chalcogenic hybrid nanostructure may be represented by As₂₋ₓCdₓS₃ (0<x<2).

According to a specific embodiment of the present disclosure, in the preparation of the second hybrid nanostructure, the chalcogen element-containing electron acceptor may be a salt of Se, and the prepared chalcogenic hybrid nanostructure may be a quaternary nanostructure comprising As, Cd, S and Se.

According to a specific embodiment of the present disclosure, in the preparation of the second hybrid nanostructure, either or both of the chalcogenic hybrid nanostructure and the second chalcogenic hybrid nanostructure may be a nanotube or a nanowire.

According to a specific embodiment of the present disclosure, in the preparation of the second hybrid nanostructure, the second chalcogenic hybrid nanostructure may be represented by As₂₋ₓCdxS_{3-y}Se_{y} (0<x<2, 0<y<3).

In another aspect, the present disclosure provides a chalcogenic hybrid nanostructure prepared by one of the afore-described methods.

Since the chalcogenic hybrid nanostructure is prepared by the afore-described methods, detailed description thereof will be omitted to avoid unnecessarily obscuring the present disclosure.

According to a specific embodiment of the present disclosure, the chalcogenic hybrid nanostructure may be represented by As₂SₓSe₃₋ₓ (0<x<3), As₂₋ₓCdₓS₃ (0<x<2) or As₂₋ₓCdxS_{3-y}Se_{y} (0<x<2, 0<y<3).

The features and advantages of the present disclosure may be summarized as follows:

(a) The present disclosure provides a new method allowing preparation of a chalcogenic hybrid nanostructure comprising three or more components using metal-reducing bacteria.

(b) The present disclosure allows preparation of a nanostructure in a more economical and eco-friendly manner.

(c) The present disclosure allows control of morphological, physical/chemical and electrical properties of the prepared nanostructure.

(d) The present disclosure provides a nanomaterial that can be useful in nanoelectronic and optoelectronic devices.

While the present disclosure has been described with respect to the specific embodiments, it will be apparent to those skilled in the art that various changes and modifications may be made without departing from the spirit and scope of the disclosure as defined in the following claims.

### EXAMPLES

### Materials and Methods

### Formation of Ternary As—S—Se, and As—Cd—S, and Quaternary As—Cd—S—Se Nanotubes

The As—S nanotubes were produced by Shewanella sp. HN-41 in the dark at 30° C. for 7 days as previously described⁴. The nanotubes were collected from culture medium, washed three times in anaerobic deionized water, and then injected into the HEPES-buffered basal medium²⁷ which supplemented with 10 mM sodium lactate as the electron donor and 2 mM sodium selenite as the electron acceptor to produce the ternary As—S—Se nanotubes. Inoculation of bacteria was performed in the same way as producing As—S, followed by incubation in the dark at 30° C. for 24 hr. In contrast, the ternary As—Cd—S nanotubes were produced through an abiotic galvanic displacement reaction. The As—S nanotubes were washed in anaerobic deionized water for 3 times, followed by resuspending in N₂-purged 2 mM CdCl₂ solution. The reaction was performed under the dark at 30° C. with gently shaking for 2 hr. The quaternary As—Cd—S—Se nanotubes were biologically synthesized by using the purified As—Cd—S nanotubes as the precursor under the same conditions as used for the synthesis of the ternary As—S—Se nanotubes.

The samples were collected at selected time during the microbial and abiotic reactions for the detection of arsenic, sulfide, selenite and Cd(II) in the aqueous reaction solutions. Culture supernatants were filtered through a 0.2 μm membrane filter (MFS-25, Advantec MFS, Inc., Dublin, Calif.), and the filtrates were diluted and acidified with 2% HNO₃ for analysis using inductively-coupled plasma mass spectrometry (ICP-MS, Agilent Technologies 7500ce, Palo Alto, Calif.). The concentration of sulfide in aqueous phase was determined by the methylene blue method²⁸. On the other hand, in order to verify the formation and the composition, the nanotubes were collected from the vessels during the reaction and then dissolved in 60% HNO₃. Content of As, Cd and Se was also detected by ICP-MS as described above.

### Material Characterization

The morphology of the nanotubes was examined by using scanning and transmission, and high resolution transmission electron microscope (SEM, TEM, and HR-TEM). SEM and TEM images were obtained using a Hitachi S-4700 FE-SEM (Tokyo, Japan) and Jeol JEM-2100F (Tokyo, Japan), respectively. SAED (selected area electron diffraction) and FFT (Fast Fourier Transform) analyses were conducted using the HR-TEM to determine crystal structures and grain size. Spatial resolved elemental analyses of cross sections of the nanotubes were done by using FE-TEM in Korea Basic Science Institute (KBSI, Daejeon, Korea). The crystal structure of the nanotubes was investigated by using X-ray diffraction (XRD, D/MAX Uitima Ill, Rigaku, Tokyo, Japan).

### Electrical Characterization

Electrode arrays were microfabricated as described previously²⁹ on silicon substrate using standard lithographic patterning. Approximately 100 nm thick SiO₂ film was first deposited on a highly doped p-type (100) oriented Si wafer using thermal chemical vapor deposition (CVD) to insulate the substrate. The electrode area was defined by photolithography using positive photoresist, followed by e-beam evaporation of a 200 Å-thick Cr adhesion layer and a ˜1800 Å-thick gold layer. Finally, electrodes (200 μm×200 μm) separated by a gap of approximately 3 μm were defined using lift-off techniques.

To fabricate nanotubes network interconnects across the electrodes; first, synthesized nanotubes were dispersed in deionized water. Then, a 3 μl drop of the nanotubes suspension solution was manually dispensed on top of the electrode gap using a micro syringe, followed by applying AC dielectrophoretic field of Vᵣₘₛ=0.36 V at f=4 MHz. After assembly, the devices were rinsed with deionized water, dried by gently blowing of nitrogen gas. To reduce the contact resistance between the electrodes and nanotubes, the samples were annealed at 100° C. for 10 min in ambient environments. The temperature dependent current-voltage (I-V) characteristics were measured using a single-channel system source meter instrument (Keithley, Model 236, Cleveland, Ohio) with various of temperature from 0 to 270K using cold-finger cryogenic system (Janis CCS-350SH). Activation energies (E_{A}) were calculated from electrical resistance Arrhenius plots in the temperature region above 210 K. The field-effect transistor transfer characteristics were measured by using the highly doped Si substrate as a back gate. The electrical measurements were performed using a dual-channel system sourcemeter instrument (Keithley 2636, Cleveland, Ohio) in ambient environments and at room temperature.

### Results and Discussion

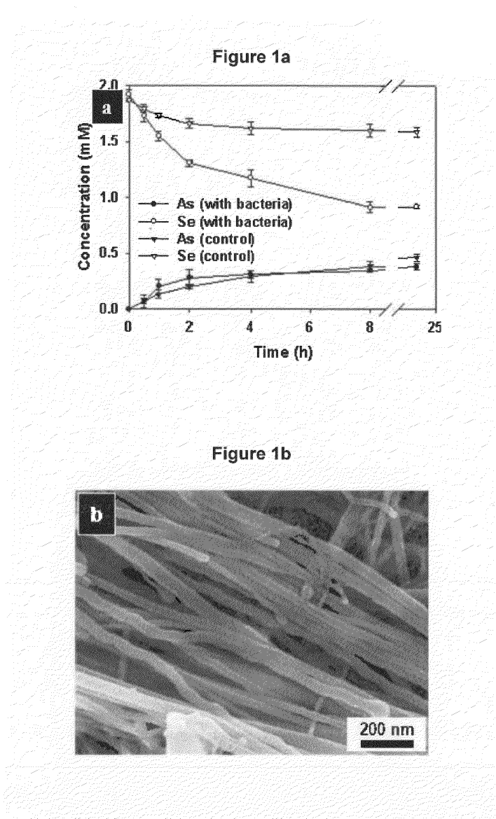

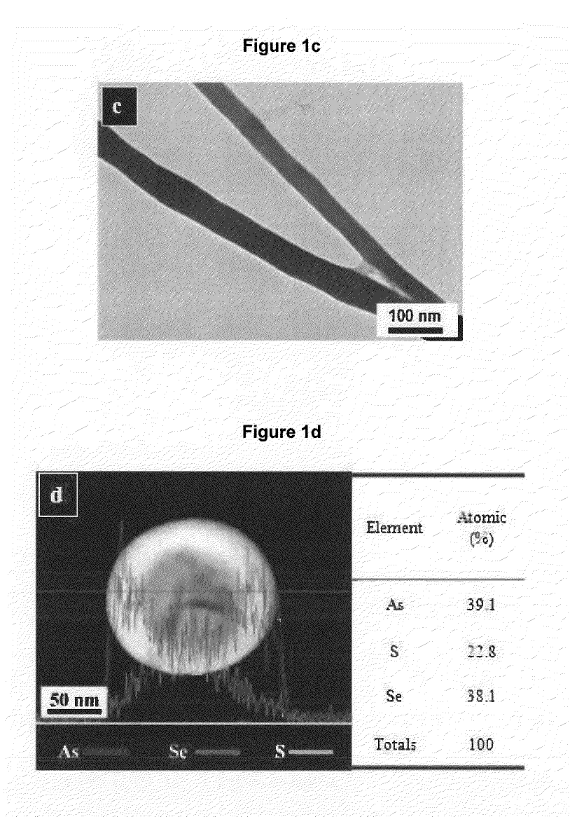

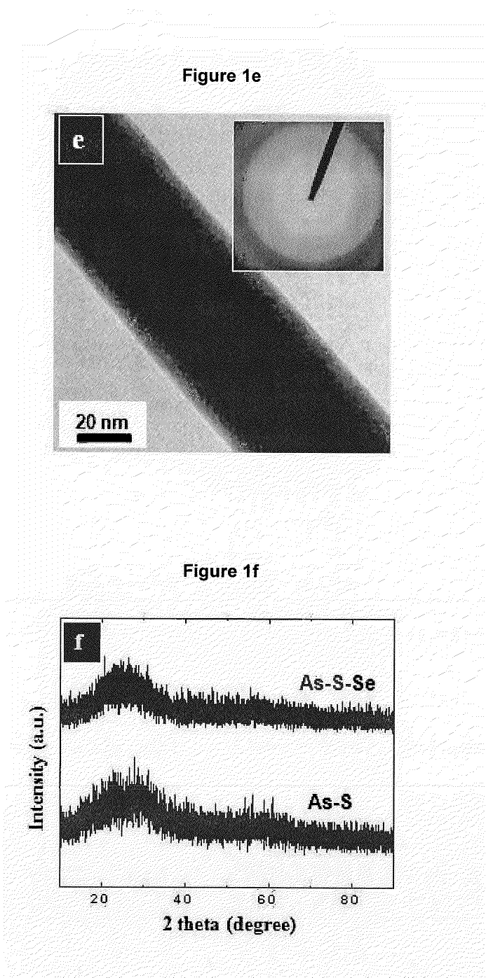

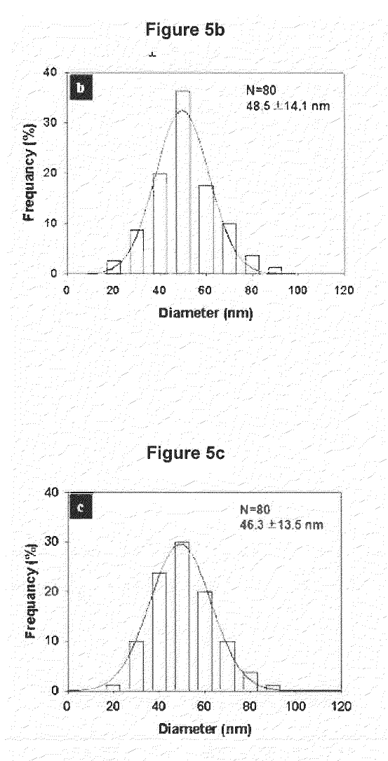

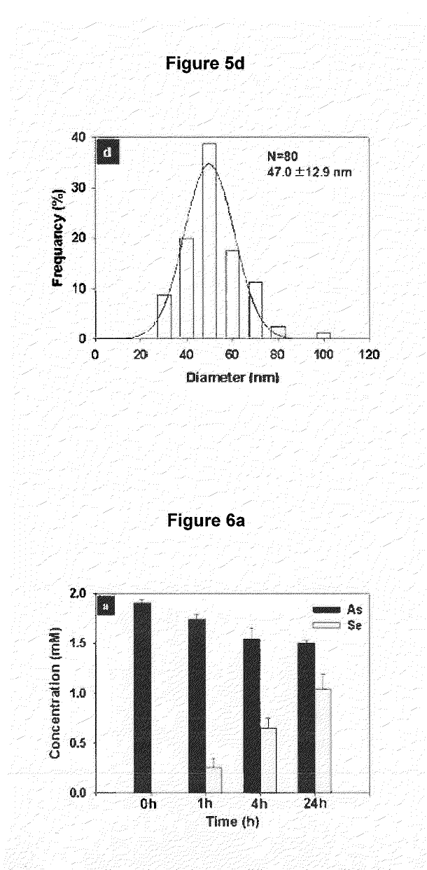

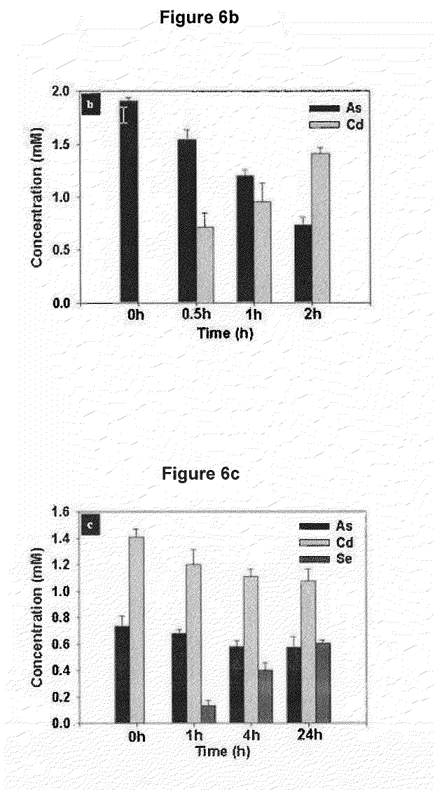

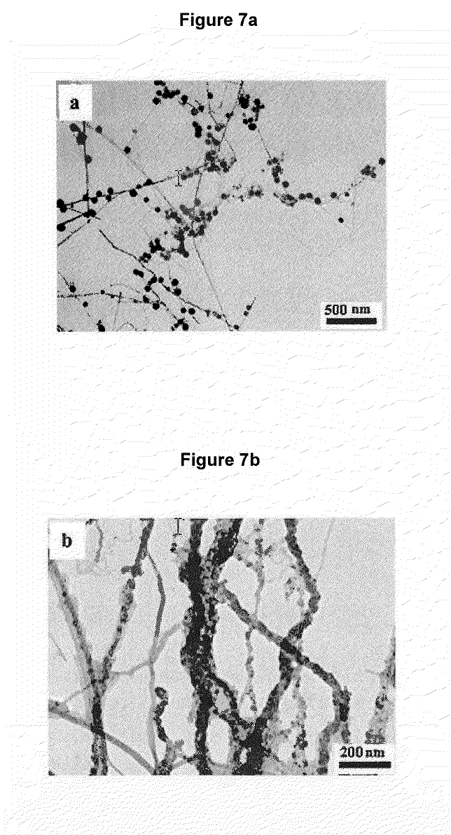

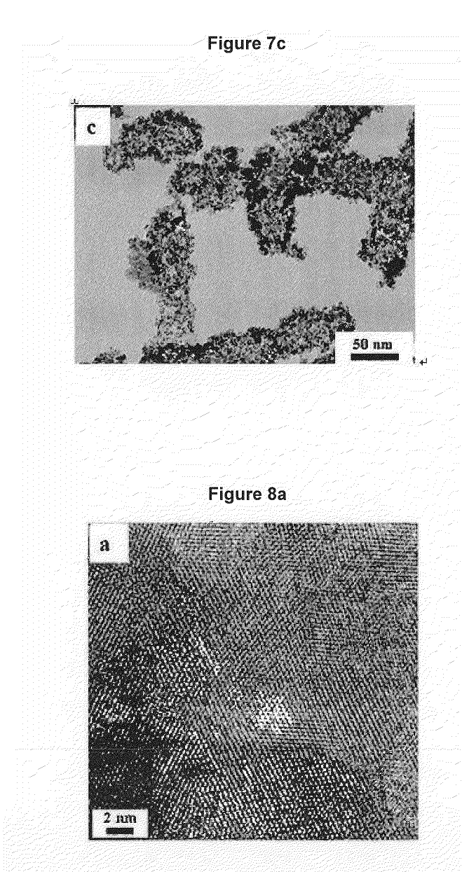

Shewanella strain HN-41 produced the As—S nanotubes via concomitant reduction of As(V) to As(III) and S₂O₃²⁻ to S²⁻ when both 5 mM As(V) and 5 mM thiosulfate were present in the anaerobic medium. Measurement of the total arsenic remained in the solution phase suggested that about 2 mM arsenic was precipitated as the As—S nanotubes after 7 d incubation (data not shown). The purified bright yellow As—S nanotubes were resuspended in the same medium supplemented with 10 mM lactate and 2 mM sodium selenite as the electron donor and acceptor, respectively. After 24 h incubation with the bacterial inoculum, the concentration of dissolved Se in the culture decreased from 2 to 0.9 mM (FIG. 1a). However, the concentration of Se did not change in the medium containing the purified As—S nanotubes in the absence of bacteria. The morphology of the red color precipitates, which were formed after incubation, was characterized by SEM and TEM microscopy. The nanostructures were composed of filamentous structures with smooth surface morphology (FIG. 1b and c) and the average diameter was approximately 48±14 nm (FIG. 5b) similar to the precursor As—S nanotubes (FIG. 5a). Cross-sectional TEM images show tube features similar to the As—S nanotubes. EDX line spectral analysis showed the presence of As, S and Se in a ratio of 2:1:2 (FIG. 1d) whereas the ratio of As:S in the As—S nanotubes was 2:3, suggesting that the S in the As—S nanotubes was replaced by Se. The similar diameter of the As—S—Se nanotubes in comparison with the As—S also indirectly proved that the synthesis of As—S—Se nanotubes was a replacement reaction rather than deposition. Measurement of the As and Se in the HNO₃-digested As—S—Se nanotubes by ICP-MS showed that the nanotubes contain considerable amount of Se (FIG. 6a) where the incorporated Se content increased with an increase in reaction time. These results implied that the composition of the As—S—Se nanotubes appeared to be an ion-exchange reaction and can be tunable as As₂SₓSe₃₋ₓ depends on different reaction rate. The control experiment showed that no As—S—Se nanotubes were formed in the media without bacteria (FIG. 7a), suggesting that the synthesis of the As—S—Se was a biological process. However, since we previously reported that Shewanella sp. HN-41 cannot reduce Se (IV) to Se (-II) but only to Se (0)⁴, formation of the Se (-II) is not clearly understood yet. On the other hand, when Se(IV) was added to the culture medium while Shewanella sp. HN-41 producing the As—S nanotubes, irregular structures of the As—S nanotubes attached by nodules-like elemental Se were formed (FIG. 7b). It seemed that Se(IV) was rapidly reduced to elemental Se by remained sulfide in the liquid culture, suggesting no displacement of sulfur with Se. The XRD pattern of the nanotubes showed a broad peak with no distinct peaks, indicating that the nanotubes were amorphous (FIG. 1f) similar to the precursor As—S nanotubes. SAED diffraction patterns also confirmed the results (FIG. 1e).

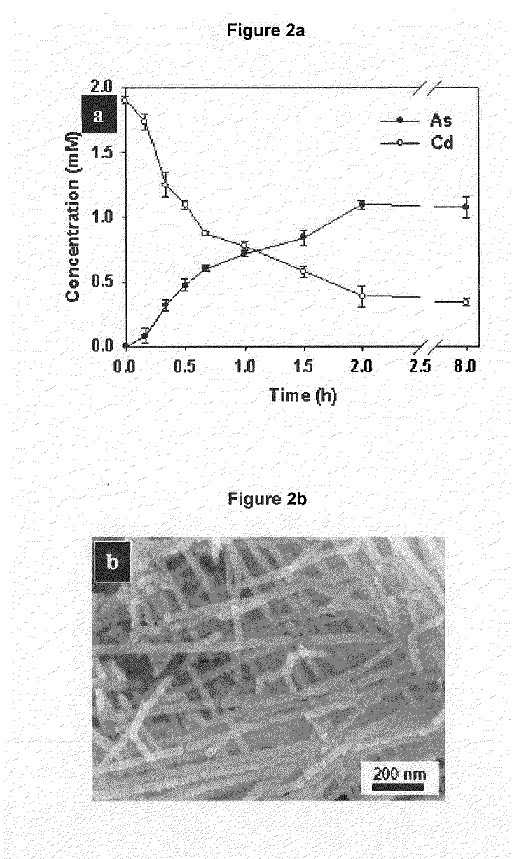

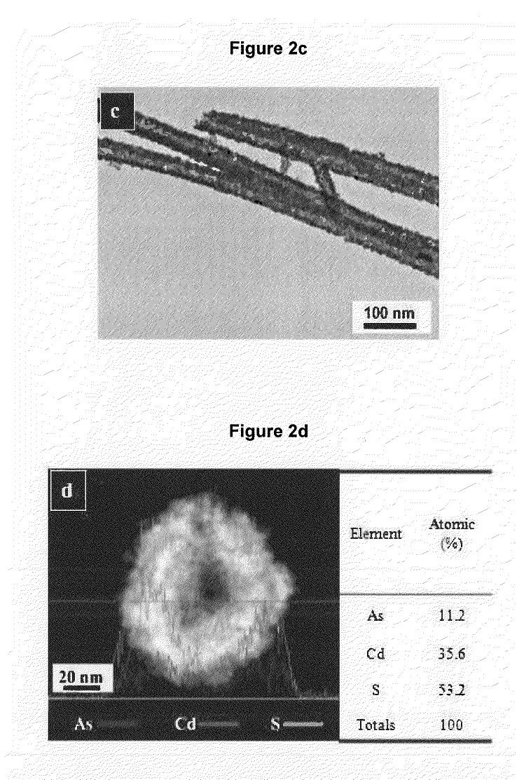

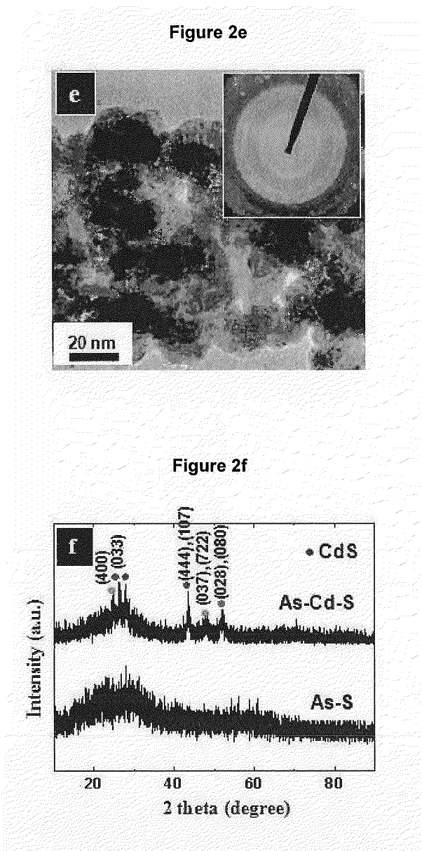

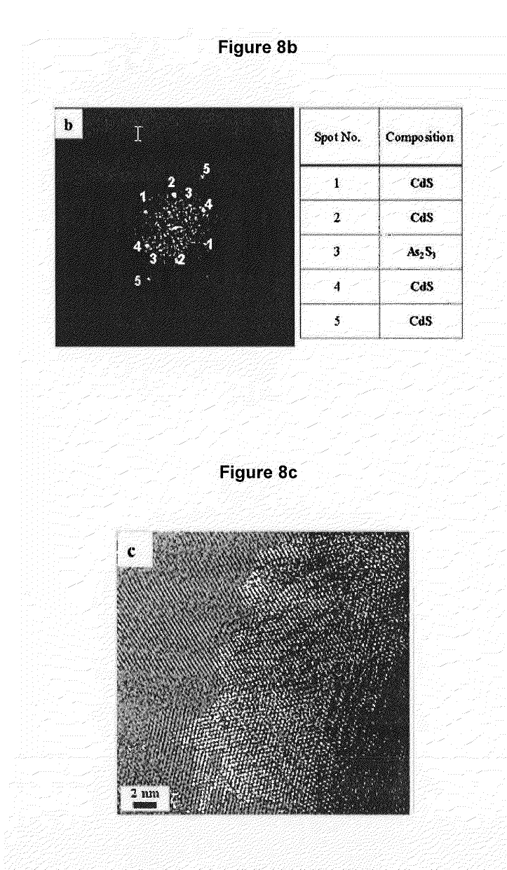

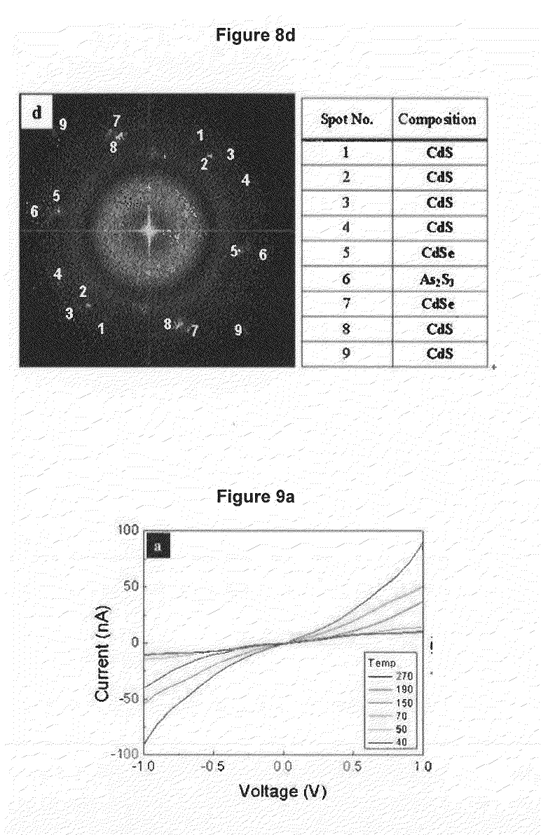

In contrast to As—S—Se, the As—Cd—S nanotubes were synthesized through an abiotic process. The purified As—S nanotubes, which were formed previously in 100 ml medium, were resuspended in the same volume containing 2 mM CdCl₂. As the reaction time increased, the color of the bright yellow As—S nanotubes changed to jacinthe. The concentration of Cd in the liquid phase decreased from 2 to 0.4 mM and As increased from 0 mM to 1.1 mM (FIG. 2a) after 2 hr incubation. The SEM and TEM images of the precipitates collected after 2 hr reaction revealed that the nanostructures maintained filamentous structures with rough surface morphology (FIGS. 2b and c). However, the structure became fragile and unstable as the reaction time increased up to 8 hr (FIG. 7c). The average diameter of the filamentous was 46±13 nm (FIG. 5c) also close to that of the As—S nanotubes. Tubular structure has been observed by cross-sectional TEM images. EDX line spectral analysis showed the ratio of As, Cd and S was approximately 1:4:5 (FIG. 2d). Compared to the composition of the As—S nanotubes, the ratio of As to S in the As—Cd—S nanotubes was significantly lower. The results of ICP-MS analysis in the incubation solution and EDX suggested that As was likely replaced by Cd. It was also confirmed by measurement of decreased As, and increased Cd in the As—Cd—S nanotubes with reaction time (FIG. 6b). These results indicated that Cd incorporation into the As—S nanotubes via cation exchange reaction was tunable by controlling the reaction time. XRD spectral analysis showed that several diffraction peaks of CdS with the preferred crystal orientation in the (444) and (107) direction (FIG. 2f). The calculated average grain size of CdS by Scherrer formula was approximately 13 nm. Although the As—S phase was not observed in the XRD pattern of the As—Cd—S nanotubes, As₂S₃ phase in the nanotubes was observed in SAED (FIG. 2e) and FFT (FIG. 8b) analysis, indicating that a small amount of As₂S₃ co-existed with CdS in the As—Cd—S nanotubes.

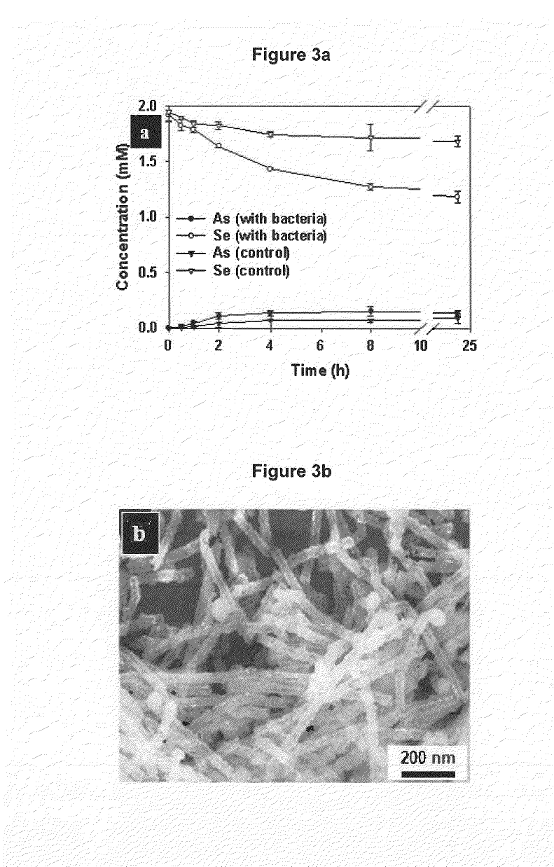

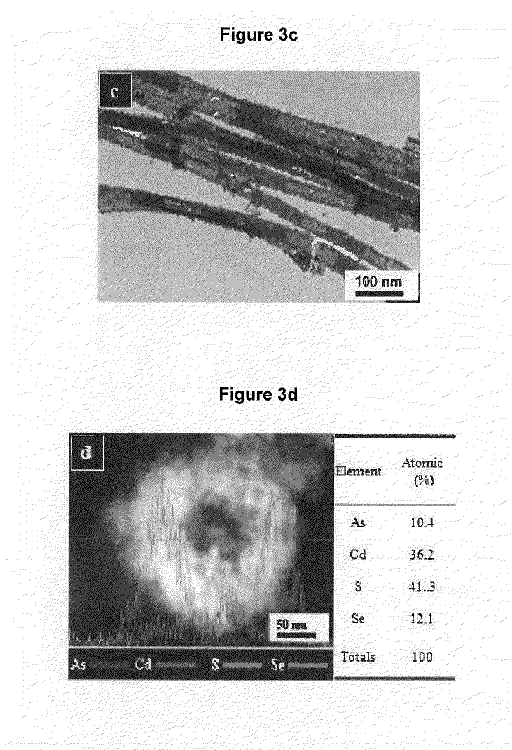

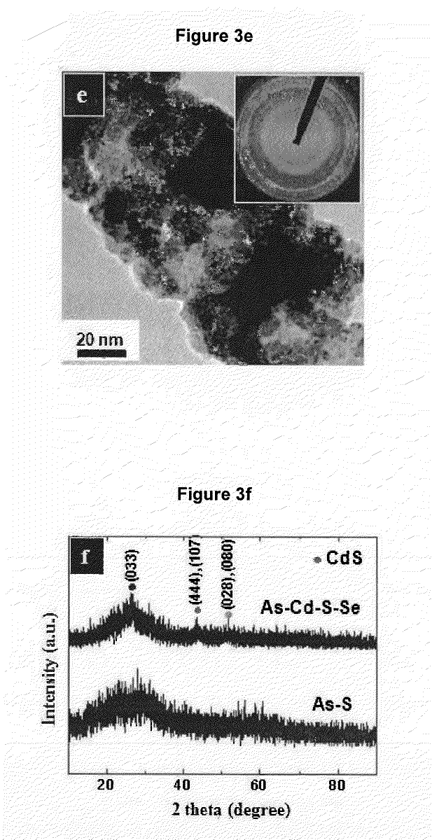

To synthesize quaternary nanotubes, the As—Cd—S was purified and resuspended in the same medium containing bacteria and 2 mM Se(IV) as described above. After 24 hr incubation, the color of the orange As—Cd—S changed to red color similar to the ternary As—S—Se nanotube. The concentration of Se in the liquid phase decreased from 2 to 1.2 mM (FIG. 3a) while Cd and As concentrations were not changed. As compared to the medium with bacteria, the concentration of Se was not significantly decreased in the medium without bacteria. The SEM and TEM images revealed that the filamentous nanostructures with rough surface were similar to the As—Cd—S nanotubes (FIGS. 3b and c). The average diameter of the filamentous was 47±13 nm, which was similar to that of the As—Cd—S nanotubes (FIG. 5d). Cross sectional TEM images showed tubular structures and EDX line spectral analysis showed that the ratio of As, Cd, S and Se was about 1:4:4:1 (FIG. 3d). Detection of As, Cd and Se in the quaternary As—Cd—S—Se nanotubes by ICP-MS indicated that the nanotubes contained considerable amounts of Cd and Se (FIG. 6c). The line-scan EDX profile of the cross section sample showed incorporation of small amount of Se bonded to As in the central region of the As—Cd—S nanotubes. Furthermore, the XRD spectra showed several diffraction peaks assigned to CdS with no peaks corresponding to CdSe and AsSe (FIG. 3f). The preferred crystal planes of CdS in the As—Cd—S—Se nanotubes were (444) and (107) which is similar to the As—Cd—S nanotubes. In the result of SAED pattern (FIG. 3e), the analyzed compositions were also similar to those of the As—Cd—S nanotubes. The results suggest that after Cd replaces As ion in the synthesis of the ternary As—Cd—S nanotubes from the biogenic As—S nanotubes, majority of S was present as CdS stably, which cannot be easily replaced by Se in the biological process of synthesizing the quaternary As—Cd—S—Se nanotubes from the As—Cd—S nanotubes. Thus the subsequent Se ion-exchange predominantly occurred in the central region where a small amount of As—S was remained. The grain size of CdS in the As—Cd—S—Se nanotubes was approximately 2.4 nm. On the other hand, CdSe was observed with CdS and As₂S₃ in the FFT analysis (FIG. 8d) of the As—Cd—S—Se nanotubes.

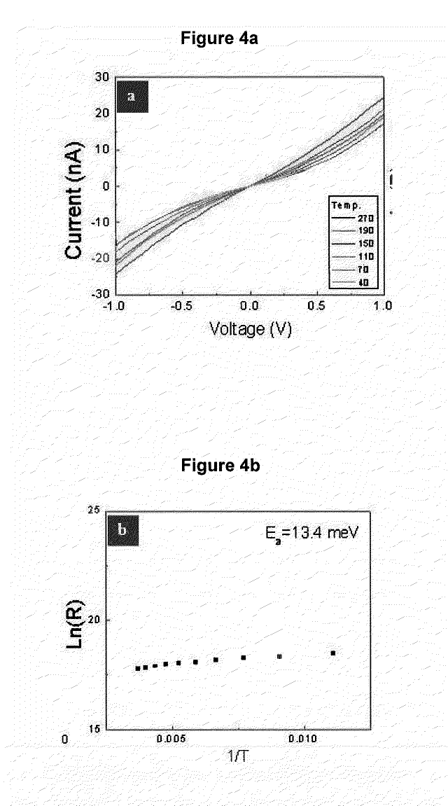

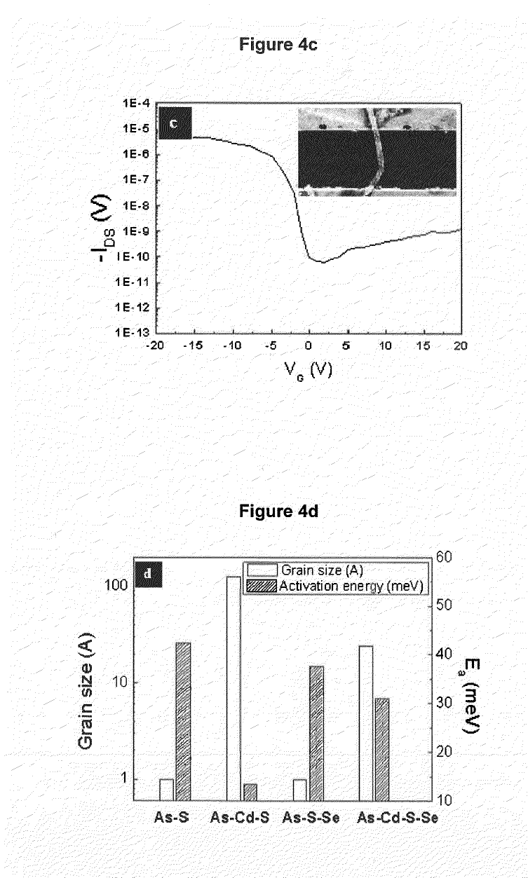

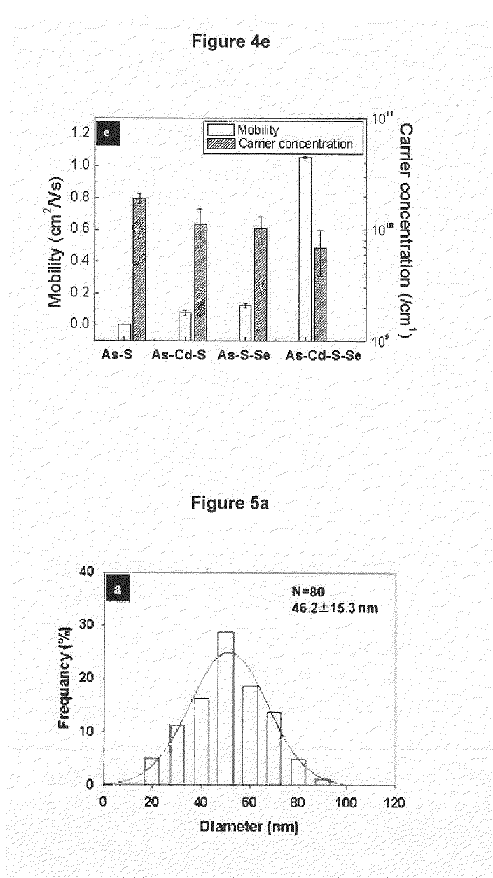

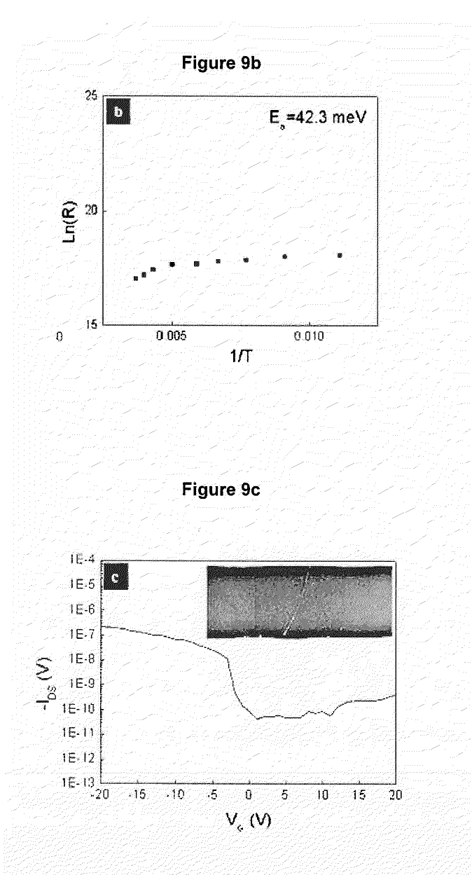

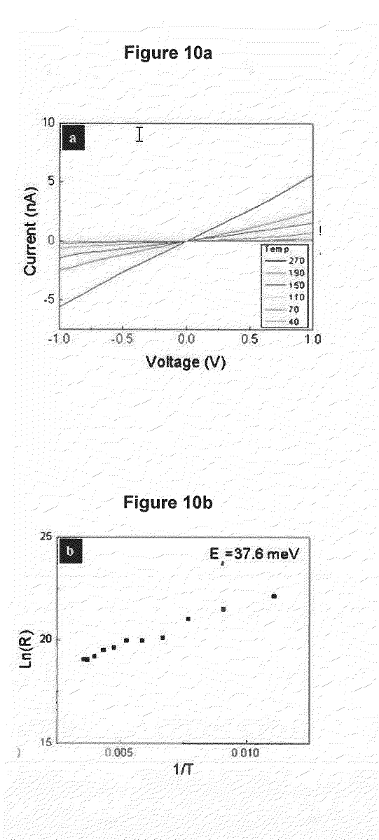

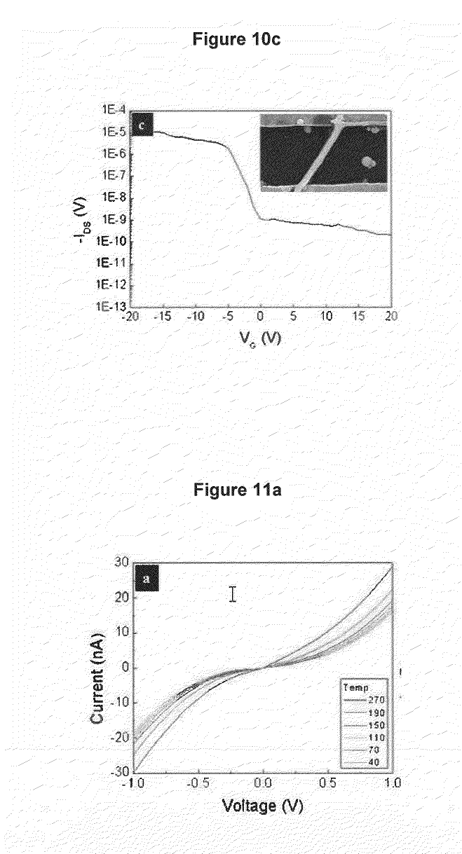

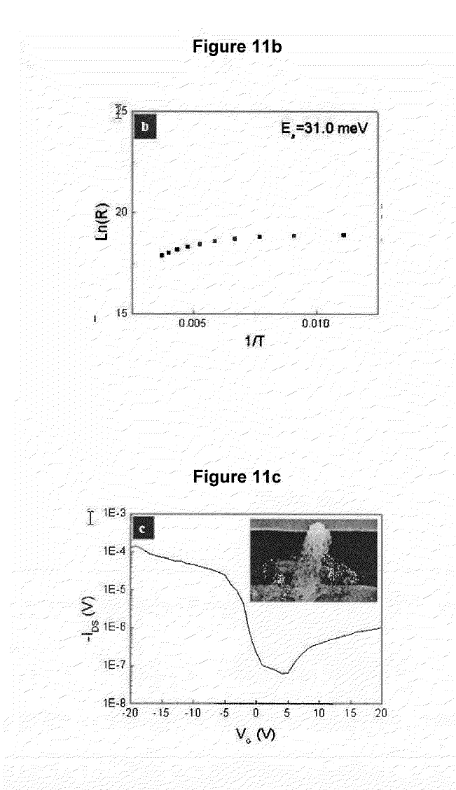

FIG. 4a, b and c showed typical I-V characteristics of single As—Cd—S nanotubes assembled across gold electrodes. The electrical properties of the As—S, As—S—Se, and As—Cd—S—Se nanotubes are shown in FIGS. 9, 10, and 11, respectively. At 270K, As—Cd—S network showed almost linear I-V characteristics (FIG. 4a) which indicated that the As—Cd—S nanotubes formed an ohmic contact. However, as the temperature decreased, the I-V curves became non-linear which might be caused by the decrease of carrier concentration resulting from lower tunneling probability. FIG. 4b shows the temperature dependent resistance which can be described by the following equation

$$R = {R}_{0} exp ( \frac{{E}_{A}}{kT} ) \qquad Eq . ( 1 )$$

where E_{A} is the conduction activation energy and R₀ is the pre-exponential factor of the resistance. The small activation energy of 13.4 meV was obtained from 270 to 210K from the As—Cd—S nanotubes which implied a low density of deep charge traps and subsequent high channel conductivity. To further investigate of electrical properties, FET transfer characteristics were measured (FIG. 4c). The carrier concentration and field effect mobility were estimated using following equations:

p=C_{G}V_{G,T}/eL_{SD} Eq. (2)

μ=L²_{SD}dI/dV/C_{G}V_{D} Eq. (3)

C_{G}=∈WL_{SD}/L_{OX} Eq. (4)

where p is the hole carrier concentration, C_{G} the approximate capacitance, V_{G,T} the threshold voltage to deplete the nanotubes, μ the field effect carrier mobility, V_{D} the drain voltage, and ∈ the dielectric constant of SiO₂²⁴. The transconductance of dI/dV was taken from each transfer characteristics in the linear regime to calculate the field effect mobility of μ. As shown in the FIG. 4c, the source-drain current (I_{DS}) was strongly dependent on the gate bias where a clear off-state at positive bias. These results infer that the As—Cd—S nanotubes are p-type semiconductor with the carrier concentration and field effect mobility of 1.1±0.4×10¹⁰ cm⁻¹ and 0.08±0.01 cm²/Vs, respectively. Inset in FIG. 4c shows the SEM image of assembled single As—Cd—S nanotubes.

FIG. 4d and e shows comparison of grain size, thermal activation energy, carrier concentration, and field effect mobility among the As—S, As—Cd—S, As—Se—S and Cd—As—Se—S nanotubes. The conduction of the nanotubes was governed by the grain boundary scattering where the amorphous/nanocrystalline As—S and As—S—Se nanotubes have much lower carrier concentration and mobility than the single or polycrystalline As—Cd—S and As—Cd—Se—S nanotubes. As expected, we found that the nanocrystalline As—Cd—S and As—Cd—Se—S nanotubes have lower thermal activation energy, E_{A}, than the amorphous As—S and As—Se—S nanotubes (FIG. 4d).

If interface states and bound charges at gate dielectric/nanotubes are absent, the concentration of the carriers and field effect mobility are mainly controlled by structure of the nanotubes and the superposition of gate electric field. Even though the carrier concentration of all nanotubes is around 10¹⁰ cm⁻¹, the field effect mobility was strongly depended on the composition of the nanotubes. For example, the quaternary As—Cd—S—Se nanotubes show highest field effect mobility, indicating that it has lowest interface states among them (FIG. 4e). These results revealed that the incorporation of Cd and/or Se into the As—S nanotubes could tune both structural and electrical properties.

In summary, chemical composition of the biogenic photoactive As—S nanotubes can be tuned by biological and abiological processes, producing the chalcogenide ternary and quaternary nanotubes by incorporation of Cd and/or Se into their nanotubes structures. Compared to the classic important techniques for synthesis of nanostructures such as thermo-facilitated Kirkendall effect^{25, 26} and cation exchange reaction^{19, 20}, this versatile, rapid, conditional, selective, and dose-dependent synthetic ability to construct and transform the biologically-originated As—S nanotubes can provide new opportunities to develop composition and structure dependent nanomaterials and tune their chemical/physical properties, which ultimately may find use in novel nano- and opto-electronic devices.

### REFERENCE

1. Hu, J. et al. Linearly polarized emission from colloidal semiconductor quantum rods. Science 292, 2060-2063 (2001).
2. Trindade, T., O'Brien, P. & Pickett, N. L. Nanocrystalline semiconductors: synthesis, properties, and perspectives. Chem. Mater. 13, 3843-3858 (2001).
3. Iijima, S. Helical microtubules of graphitic carbon. Nature 354, 56-58 (1991).
4. Lee, J.-H. et al. Biogenic formation of photoactive arsenic-sulfide nanotubes by Shewanella sp. HN-41. Proc. Natl. Acad. Sci. USA 104, 20410-20415 (2007).
5. Sapra, S., Sarma, D. D., Sanvito, S. & Hill, N. A. Influence of quantum confinement on the electronic and magnetic properties of (Ga,Mn)As diluted magnetic semiconductor. Nano Lett. 2, 605-608 (2002).
6. Rao, C. N. R. & Nath, M. Inorganic nanotubes. Dalton. Trans. 1, 1-24 (2003).
7. Chen, J. C., Lin, Z. H. & Ma, X. X. Evidence of the production of silver nanoparticles via pretreatment of Phoma sp. 3.2883 with silver nitrate. Lett. Appl. Microbiol. 37, 105-108 (2003).
8. Fortin, D. What biogenic minerals tell us. Science 303, 1618-1619 (2004).
9. Lowenstam, H. A. Minerals formed by organisms Science 211, 1126-1131 (1981).
10. Newman, D. K. How bacteria respire minerals. Science 292, 1312-1313 (2001).
11. Gorby, Y. A. & Lovley, D. R. Electron transport in the dissimilatory iron reducer, GS-15. Appl. Environ. Microbiol. 57, 867-870 (1991).
12. Lovley, D. R. Dissimilatory Fe(III) and Mn(IV) reduction. Microbiol. Rev. 55, 259-87 (1991).
13. Fredrickson, J. K., Kostandarithes, H. M., Li, S. W., Plymale, A. E. & Daly, M. J. Reduction of Fe(III), Cr(VI), U(VI), and Tc(VII) by Deinococcus radiodurans R1. Appl. Environ. Microbiol. 66, 2006-2011 (2000).
14. Dameron, C. T. et al. Biosynthesis of cadmium sulphide quantum semiconductor crystallites. Nature. 338, 596-597 (1989).
15. Lovley, D. R., Stolz, J. F., Nord, G. L. & Phillips, E. J. P. Anaerobic production of magnetite by a dissimilatory iron-reducing microorganism. Nature. 330, 252-254 (1987).
16. Lee, J. H., Han, J. H., Choi, H. C. & Hur, H.-G. Effects of temperature and dissolved oxygen on Se(IV) removal and Se(0) precipitation by Shewanella sp. HN-41. Chemosphere 68, 1898-1905 (2007).
17. Jiang, S. et al. Biogenic formation of As—S nanotubes by diverse Shewanella strains. Appl. Environ. Microbiol. 75, 6896-6899 (2009).
18. Tam, K. et al. Growth mechanism of amorphous selenium nanoparticles synthesized by Shewanella sp. HN-41. Biosci. Biotechnol. Biochem. (Accepted).
19. Son, D. H., Hughes, S. M., Yin, Y. & Paul Alivisatos, A. Cation exchange reactions in ionic nanocrystals. Science. 306, 1009-12 (2004).
20. Robinson, R. D. et al. Spontaneous superlattice formation in nanorods through partial cation exchange. Science. 317, 355-358 (2007).
21. Duan, X., Huang, Y., Agarwal, R. & Lieber, C. M. Single-nanowire electrically driven lasers. Nature. 421, 241-245 (2003).
22. Minami, T. New n-type transparent conducting oxides. MRS Bulletin 25, 38-44 (2000).
23. Kum, M. C. et al. Synthesis and characterization of cadmium telluride nanowire. Nanotechnology. 19, 325711-325718 (2008).
24. Martel, R., Schmidt, T., Shea, H. R., Hertel, T. & Avouris, P. Single- and multi-wall carbon nanotube field-effect transistors. Appl. Phys. Lett. 73, 2447-2449 (1998).
25. Yin, Y. et al. Formation of hollow nanocrystals through the nanoscale Kirkendall effect. Science. 304, 711-714 (2004).
26. Fan, J. H. et al. Monocrystalline spinel nanotube fabrication based on the Kirkendall effect. Nat. Mater. 5, 627-631 (2006).
27. Lee, J. H., Roh, Y., Kim, K. W. & Hur, H.-G. Organic acid-dependent iron mineral formation by a newly isolated iron-reducing bacterium, Shewanella sp. HN-41. Geomicrobiol. J. 24, 31-41 (2007).
28. Fogo, J. K. & Popowski, M. Spectrophotometric determination of hydrogen sulfide. Anal. Biochem. 21, 732-734 (1949).
29. Mubeen, S., Zhang, T., Yoo, B., Deshusses, M. A. & Myung, N. V. Palladium nanoparticles decorated single-walled carbon nanotube hydrogen sensor. J. Phys. Chem. C. 111, 6321-6327 (2007).

What is claimed is:

1. A method for preparing a chalcogenic hybrid nanostructure comprising:
(a) adding a chalcogenic nanostructure, an electron donor and an electron acceptor to a medium containing metal-reducing bacteria to prepare a reaction mixture, the electron acceptor comprising a chalcogen element; and
(b) performing a metal reduction reaction using the prepared reaction mixture to prepare a chalcogenic hybrid nanostructure with the chalcogen element of the electron acceptor incorporated.

2. The method according to claim 1, wherein the metal-reducing bacteria belong to the genus Shewanella.

3. The method according to claim 1, wherein the chalcogenic nanostructure comprises at least one chalcogen element selected from a group consisting of As, Cd, Zn, S, Se and Te.

4. The method according to claim 3, wherein the chalcogenic nanostructure is a binary nanostructure comprising As and S.

5. The method according to claim 1, wherein the electron acceptor comprising a chalcogen element is a salt comprising a chalcogen element in oxidized state.

6. The method according to claim 4, wherein the electron acceptor comprising a chalcogen element is a salt of Se, and the prepared chalcogenic hybrid nanostructure is a ternary nanostructure comprising As, S and Se.

7. The method according to claim 1, wherein either or both of the chalcogenic nanostructure and the chalcogenic hybrid nanostructure is(are) a nanotube or a nanowire.

8. The method according to claim 1, wherein the chalcogen element of the electron acceptor is incorporated into the chalcogenic nanostructure through replacement rather than through deposition.

9. The method according to claim 4, wherein the chalcogenic nanostructure is a binary nanostructure comprising As and S, and the chalcogen element of the electron acceptor is incorporated into the chalcogenic nanostructure by partially replacing S through replacement rather than through deposition.

10. The method according to claim 9, wherein the chalcogenic hybrid nanostructure is represented by As₂SₓSe₃₋ₓ (0<x<3).

11. A method for preparing a chalcogenic hybrid nanostructure comprising:
preparing a reaction mixture comprising a chalcogenic nanostructure and a chalcogen element-containing salt; and
performing an ion-exchange reaction using the prepared reaction mixture to prepare a chalcogenic hybrid nanostructure with the chalcogen element of the chalcogen element-containing salt incorporated.

12. The method according to claim 1, wherein the chalcogenic nanostructure is one synthesized biogenically using metal-reducing bacteria.

13. The method according to claim 1, wherein the chalcogenic nanostructure comprises at least one chalcogen element selected from a group consisting of As, Cd, Zn, S, Se and Te.

14. The method according to claim 13, wherein the chalcogenic nanostructure is a binary nanostructure comprising As and S.

15. The method according to claim 11, wherein the chalcogen element-containing salt is a salt comprising a chalcogen element in oxidized state.

16. The method according to claim 14, wherein the chalcogen element-containing salt is a salt of Cd, and the prepared chalcogenic hybrid nanostructure is a ternary nanostructure comprising As, Cd and S.

17. The method according to claim 11, wherein either or both of the chalcogenic nanostructure and the chalcogenic hybrid nanostructure is(are) a nanotube or a nanowire.

18. The method according to claim 14, wherein the chalcogenic nanostructure is a binary nanostructure comprising As and S, and the chalcogen element of the chalcogen element-containing salt is incorporated into the chalcogenic nanostructure by partially replacing As through cation-exchange reaction.

19. The method according to claim 18, wherein the chalcogenic hybrid nanostructure is represented by As₂₋ₓCdₓS₃ (0<x<2).

20. The method according to claim 19, wherein the chalcogenic hybrid nanostructure represented by As₂₋ₓCdₓS₃ (0<x<2) has p-type semiconductor properties.

21. The method according to claim 1, which further comprises, after performing the metal reduction reaction: adding a medium containing metal-reducing bacteria, an electron donor and a chalcogen element-containing electron acceptor to the prepared chalcogenic hybrid nanostructure to prepare a reaction mixture; and performing a metal reduction reaction to prepare a second chalcogenic hybrid nanostructure with the chalcogen element of the electron acceptor incorporated.

22. The method according to claim 11, which further comprises, after performing the ion-exchange reaction: adding a medium containing metal-reducing bacteria, an electron donor and a chalcogen element-containing electron acceptor to the prepared chalcogenic hybrid nanostructure to prepare a reaction mixture; and performing a metal reduction reaction to prepare a second chalcogenic hybrid nanostructure with the chalcogen element of the electron acceptor incorporated.

23. The method according to claim 22, wherein the chalcogenic hybrid nanostructure is represented by As₂₋ₓCdₓS₃ (0<x<2).

24. The method according to claim 22, wherein the chalcogen element-containing electron acceptor is a salt of Se, and the prepared chalcogenic hybrid nanostructure is a quaternary nanostructure comprising As, Cd, S and Se.

25. The method according to claim 22, wherein either or both of the chalcogenic hybrid nanostructure and the second chalcogenic hybrid nanostructure is(are) a nanotube or a nanowire.

26. The method according to claim 24, wherein the second chalcogenic hybrid nanostructure is represented by As₂,CdxS_{3-y}Se_{y} (0<x<2, 0<y<3) (0<x<5, 0<y<5).

27. A chalcogenic hybrid nanostructure prepared by a method according to claim 1.

28. The chalcogenic hybrid nanostructure according to claim 27, wherein the chalcogenic hybrid nanostructure is represented by As₂SₓSe₃₋ₓ (0<x<3), As₂₋ₓCdₓS₃ (0<x<2) or As₂₋ₓCdxS_{3-y}Se_{y} (0<x<2, 0<y<3).
